(19) United States
(12) Patent Application Publication
(10) Pub. No.: US 2005/0060017 A1
(43) Pub. Date: Mar. 17, 2005

(54) Means and method for the treatment of cerebral aneurysms

(76) Inventors: Robert E. Fischell, Dayton, MD (US); Scott J.S. Fischell, Glenelg, MD (US)

Correspondence Address: Dr. Robert E. Fischell, 14600 Viburnum Drive, Dayton, MD, 21036, US

(21) Appl. No.: 10/661,888

(22) Filed: Sep. 15, 2003

(51) Int. Cl. A61F 2/06

(52) U.S. Cl. 623/1.11; 623/1.15; 623/1.34; 606/200; 623/1.43

(57) ABSTRACT

Disclosed is a system for the treatment of cerebral aneurysms using a stent and a aneurysm pocket fill structure delivery system. One embodiment of the present invention uses a highly radiopaque, drug eluting stent that is deployed with its sidewall over the ostium of the aneurysm pocket. A fill structure delivery catheter is then advanced through the patient's vascular system until the catheter's distal end is situated within the aneurysm pocket. Compressed aneurysm pocket filling structures are then pushed through the fill structure delivery catheter. As the aneurysm pocket filling structures emerge from an opening in the catheter's distal end, they promptly expand so that their minimum dimension is sufficiently large so that they cannot pass through the spaces between the struts of the stent that cover the ostium of the aneurysm pocket.

### FIELD OF USE

This invention is in the field of methods and devices for the percutaneous treatment of cerebral aneurysms.

### BACKGROUND OF THE INVENTION

Arteries in the cerebral circulation occasionally have a weakness in the arterial wall that results in a cerebral aneurysm. In the USA, the most frequent treatment for this potentially life threatening condition is a surgical intervention. Unfortunately, there is a comparatively high rate of morbidity and mortality associated with these surgeries.

An alternative treatment involves the percutaneous implantation of platinum metal coils that are inserted into the aneurysm pocket. For most cerebral aneurysms, an average of six such platinum coils is required to sufficiently fill the aneurysm pocket. This makes for a procedure that is both complex and time consuming and is also comparatively costly. Furthermore, the platinum coils do not typically fill the entire aneurysm pocket and this is one factor that results in an annual failure rate for this procedure is approximately 20%.

### SUMMARY OF THE INVENTION

The present invention provides an improved treatment for both ruptured and not ruptured cerebral aneurysms. This treatment would avoid surgery and would be simpler, faster, less costly and, mostly importantly, have a decreased failure rate as compared to the percutaneous procedure that uses platinum coils. One preferred embodiment of the present invention involves three separate medical devices, namely: 1) an arterial filter; 2) a stent (preferably a drug eluting stent); and, 3) a fill structure delivery catheter system including aneurysm pocket filling structures for placement into the aneurysm pocket. Of these three medical devices, only numbers 2) and 3) are required for treating the aneurysm. One form of an aneurysm pocket filling structure is a small, hollow, thin-walled spherical shell (which we shall call a “minisphere”). The present invention also envisions other forms of aneurysm pocket filling structures that are not thin-walled spheres but are readily compressible, generally soft plastic objects. Because the aneurysm pocket filling structures of the present invention are extremely soft and readily compressible, they can be used to fully fill (and even overfill) the aneurysm pocket without risking breakage of the delicate wall of the aneurysm pocket. Still further the present invention envisions aneurysm pocket filling structures that are composed partially or completely from a biocompatible metal. Such structures would also have to expand into the aneurysm pocket after they are delivered by a fill structure delivery system.

The drug eluting stent should be able to prevent either or both intimal hyperplasia and subacute thrombosis by the elution of an anti-proliferative drug for preventing intimal hyperplasia and/or a coating such as heparin to prevent subacute thrombosis. An ideal stent for this purpose would have both an anti-proliferative drug that elutes from the stent and a surface coating that minimizes the probability of acute or subacute thrombosis. Examples of coatings to prevent intimal hyperplasia at the site of the aneurysm are cytostatic drugs such as sirolimus and everolimus or cytotoxic drugs such as paclitaxel. For the purpose of this disclosure, all such drugs shall be termed “anti-proliferative” drugs. Examples of anti-thrombogenic drug coatings to prevent acute or subacute thrombosis are heparin and phosphorocholine.

The procedure for treating the cerebral aneurysm could advantageously begin by using conventional means to place an arterial filter within the artery to be treated. The location of the deployed filter should be just distal to the ostium (mouth) of the aneurysm pocket. It should be understood however that the present invention can be practiced without first placing a filter in the cerebral artery that is being treated. The next step is to use conventional means to deploy a stent across the ostium of the aneurysm pocket. Although conventional stents made from stainless or an L605 type of cobalt-chromium alloy could be used for this purpose, highly radiopaque stents made from a metal such as tantalum would be ideal for placement in a cerebral artery. Still further, a stent made from a memory alloy such as Nitinol could be used, especially if it utilized inserts formed from a highly radiopaque metal such as tantalum. Ideally, the stent should have a wall thickness that is less than 0.004 inches and an optimum thickness would be between 0.001 and 0.003 inches. The stent could optimally be designed to have smaller cells in the mid-section of the stent and larger cells at each end section. In this way, the ostium of the aneurysm pocket would be well covered with a minimum circular opening in the part of the stent's sidewall that covers the ostium of the aneurysm pocket. After the stent is properly placed, the stent delivery system is removed and the distal end of the guide wire that was used with the stent delivery system to deliver the stent is then placed through the side of the stent and into the aneurysm pocket. It is also envisioned that the guide wire used to deliver the stent could be removed and a special guide wire for placing the fill structure delivery catheter into the aneurysm pocket could be used. A fill structure delivery catheter is then advanced over the guide wire until the catheter's radiopaque distal end becomes situated within the aneurysm pocket. The guide wire is then removed from the catheter. A separate fill structure storage tube containing many aneurysm pocket filling structures within its interior lumen is then placed with its distal end within an “O”-ring connector that is situated at the proximal end of the fill structure delivery catheter. The fill structure delivery catheter is used to place the aneurysm pocket filling structures into the aneurysm pocket. The fill structure storage tube also has a proximal fitting that can be used to inject a high-pressure liquid for pushing the aneurysm pocket filling structures through the fill structure storage tube and through the fill structure delivery catheter into the aneurysm pocket. An alternative method for pushing the aneurysm pocket filling structures through the lumens of the fill structure storage tube and the fill structure delivery catheter is a pusher rod that is sufficiently long so that it can extend to a point near the distal end of the fill structure delivery catheter. In either case, enough aneurysm pocket filling structures should be placed into the aneurysm pocket to completely fill its volume so as to isolate the aneurysm pocket from the arterial circulation. Optimally, each aneurysm pocket filling structure is an elastic structure that is very easily compressed so that the aneurysm pocket can be overfilled by at least 10% without exerting a significant pressure on the walls of the aneurysm pocket. The portion of the stent that is deployed against the ostium of the aneurysm pocket prevents any aneurysm pocket filling structure from escaping into the arterial circulation. The presence of a structure pushed against the wall of the aneurysm pocket should encourage neointimal hyperplasia of the inner surface of the wall of the aneurysm pocket. Such increased tissue growth can serve to strengthen the wall of the aneurysm pocket to prevent any future rupturing of that wall. To prevent any aneurysm pocket filling structure from passing through between the struts of the deployed stent, the minimum dimension of each deployed aneurysm pocket filling structure must be larger than the largest opening between the stent struts at the ostium of the aneurysm pocket. This attribute of having the minimum dimension of the aneurysm pocket filling structure that is larger than the maximum opening in the sidewall of the stent that is placed at the ostium of the aneurysm pocket must be true for any shape of an aneurysm pocket filling structure. This attribute is not the case for existing platinum coils that are placed inside an aneurysm pocket that has its ostium bl;ocked by a deployed stent.

The purpose of the arterial filter that coulod be placed downstream from the ostium of the aneurysm pocket is to catch any aneurysm pocket filling structure that might inadvertently escape through the sidewall of the stent into the brain's arterial circulation. Furthermore, the filter could also prevent embolization of any aneurysm pocket filling structure that is inadvertently released into the arterial circulation because the interventional neuroradiologist failed to place the distal end of the fill structures delivery catheter into the aneurysm pocket.

The minispheres form of an aneurysm pocket filling structure is a novel design that can be used with this system for the treatment of a cerebral aneurysm. Each minisphere is a thin-walled, hollow, spherical, elastomer shell whose compressed diameter is smaller than the openings in the side wall of the stent and whose deployed diameter is at least 10% larger than the maximum opening in the side wall of the stent. Optimally, the deployed diameter of the minispheres (or the minimum dimension of any other form of aneurysm pocket filling structure) is approximately 1.1 to 5 times larger than the maximum opening in the sidewall of the stent that blocks the ostium of the aneurysm pocket. Thus any minisphere placed into the aneurysm pocket will not embolize downstream into the brain's arterial circulation. Each minisphere is optimally formed from an elastomer having a comparatively low durometer; i.e., the minispheres are easily compressed. Each minisphere also has a small hole through its spherical shell. The purpose of the hole is twofold: first, air will not be trapped inside the spherical shell when it is compressed so that the minispheres can be easily compressed to a comparatively small diameter for placement through a catheter, and second, after the compressed minispheres are released into the aneurysm pocket, they will expand and blood will be pulled into the minisphere. As time passes, the blood that is sucked into the minispheres will clot thus forming a comparatively firm structure within the aneurysm pocket. This firm structure can isolate the aneurysm pocket from the blood that is flowing in the cerebral circulation. This will prevent the aneurysm pocket from continuing to increase in size. Such a size increase can result in unwanted pressure on the brain, or even worse, the aneurysm pocket can rupture causing considerable morbidity and mortality.

The thin-walled shell with a hole type of construction allows easy compression of the minispheres from their deployed diameter to a much smaller compressed diameter for placement into the lumen of the fill structure storage tube. The ratio of deployed minisphere diameter to the compressed diameter should be at least 1.1 to 1.0 and optimally between 1.5:1 and 5:1. The outer surface of each minisphere as well as the interior surfaces of the fill structure storage tube and the fill structure delivery catheter can each have a lubricious coating (such as PTFE) for decreasing the force required to push the minispheres through the fill structure storage tube and the fill structure delivery catheter. The outer surface of the minispheres could include a surface treatment to reduce thrombus formation and the inner surface of the minispheres could have no surface treatment or a surface treatment to promote blood coagulation.

An important aspect of this novel concept for the percutaneous treatment of a cerebral aneurysm is to calculate the volume of the aneurysm pocket so that a sufficient number of aneurysm pocket filling structures are placed into that pocket. This calculation of the volume of the aneurysm pocket can be made with the assistance of steriotatic image intensified fluoroscopy when the aneurysm pocket is filled with a contrast medium. Because the hollow, thin-walled, elastomer aneurysm pocket filling structures are designed to be easily compressed, it is possible and even desirable to somewhat overfill the aneurysm pocket with aneurysm pocket filling structures. For example, overfilling the volume of the aneurysm pocket by 5-20% would guarantee an adequate filling to prevent failure of the treatment. Also such overfilling would encourage tissue growth of the wall of the aneurysm pocket. When the aneurysm pocket is overfilled, at least many of the aneurysm pocket filling structures will be somewhat compressed without exerting an excessive pressure on the walls of the aneurysm pocket. It is most important to not significantly increase the pressure on the typically thin walls of the aneurysm pocket. Underfilling by more than 5% would be undesirable because it could result in an increased probability of failure and therefore should be avoided. By adding a radiopaque material to the substance from which the aneurysm pocket filling structures are formed, it is possible by fluoroscopy to determine that the aneurysm pocket is fully filled and that no aneurysm pocket filling structure has escaped into the arterial circulation. Also adding a highly radiopaque metal, e.g., in the form of a powder, can also be used to increase the radiopacity of the aneurysm pocket filling structures.

When the minispheres are placed into the fill structure storage tube prior to attachment of the fill structure storage tube into the “O”-ring connector of the fill structure delivery catheter, they are compressed and the air is pushed out through the hole in the minisphere's shell. This design feature allows the minispheres to be readily compressed without exerting a high force against the lumens of either the fill structure storage tube or the fill structure delivery catheter. This decreased force also decreases the force required to deliver the minispheres into the aneurysm pocket. The minispheres are ideally made as hollow spherical shells formed from a comparatively low durometer elastomer or even from an open-cell elastomer foam.

It is also envisioned that any aneurysm pocket filling structure can be formed from a closed-cell or open-cell elastomer foam.

Furthermore, elastic objects other than minispheres could readily be used to fill the aneurysm pocket. For example, elastomer tubes that form a torroidal shape after deployment into the aneurysm pocket could also be used for filling the aneurysm pocket. Another preferred embodiment of the present invention is to use an aneurysm pocket filling structure that is a compressed metal or plastic helix that expands radially outward after it enters the aneurysm pocket. The present invention envisions any readily compressible elastomer or metal (or combination) structure having a compressed pre-deployment shape that can be placed through a lumen of a catheter into the aneurysm pocket as being an “aneurysm pocket filling structure”.

Another requirement of such an aneurysm pocket filling structure would be that it has no dimension when deployed into the aneurysm pocket that is smaller than the largest opening between the struts of the stent that is deployed at the ostium of the aneurysm pocket

Still another preferred embodiment of the present invention is an aneurysm pocket filling structure that is formed from polyvinyl alcohol (PVA). PVA has the unique characteristic that it can be compressed to a small diameter and then placed into the lumen of a fill structure storage tube. After being placed into the storage tube, the PVA aneurysm pocket filling structures can then be exposed to a liquid that includes contrast medium. The PVA aneurysm pocket filling structures will then form an open-cell foam that is radiopaque. When released into the aneurysm pocket, this PVA form of aneurysm pocket filling structure will be extremely soft and pliable and will be able to be easily visualized with fluoroscope. The liquid that is used to fill the PVA aneurysm pocket filling structures in the fill structure storage tube can also include other drugs that could either promote or inhibit thrombus formation. For example, thrombin could be used to promote thrombogenicity and heparin could be used to decrease any tendency for creating blood clots

Ideally, the aneurysm pocket filling structures that would go to the stent surface would not promote thrombus formation and the aneurysm pocket filling structures that would not be directly exposed to the arterial blood flow would be treated to increase thrombogenicity.

Thus one object of the present invention is to close off a cerebral aneurysm by using a stent to block the mouth of the aneurysm pocket and then filling the aneurysm pocket with minispheres or any other aneurysm pocket filling structure.

Another object of this invention is to teach a comparatively simple and reliable method for the percutaneous treatment of an aneurysm in a cerebral artery.

Still another object of this invention is to first place an arterial filter into the artery that has the aneurysm to preclude the inadvertent release of an aneurysm pocket filling structure into the brain's arterial circulation.

Still another object of this invention is to utilize PVA as an aneurysm pocket filling structure.

Still another object of this invention is to slightly overfill the aneurysm pocket with extremely soft and pliable aneurysm pocket filling structures so as to promote the creation of tissue onto the wall of the aneurysm pocket.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1. is a cross section of a cerebral aneurysm formed from the wall of a cerebral artery with an arterial filter placed distal to the ostium of the aneurysm pocket, a guide wire placed into the artery and a stent deployed so as to block the ostium of the aneurysm pocket.

FIG. 2 is the cross section of FIG. 1 with the tip of the guide wire being placed into the aneurysm pocket.

FIG. 3 illustrates a fill structure delivery catheter advanced over the guide wire with the catheter's distal end placed into the aneurysm pocket.

FIG. 4 is a longitudinal cross section of a distal portion of the fill structure delivery catheter showing a tapered and slotted end section.

FIG. 5 illustrates the guide wire being removed from the fill structure delivery catheter, three minispheres deployed into the aneurysm pocket and one minisphere partially emerging from the distal end of the fill structure delivery catheter.

FIG. 6 is a highly enlarged cross section of a hollow shell minisphere with a hole placed through the minisphere's shell.

FIG. 7 is highly enlarged cross section of a compressed minisphere as it would be shaped for passage through the lumens of the fill structure storage tube and the fill structure delivery catheter.

FIG. 8 is a longitudinal cross section of a fill structure storage tube into which a compressed minisphere is placed near the tube's proximal end and two alternative aneurysm pocket filling structures that are compressed cylinders are placed at a distal portion of the fill structure storage tube.

FIG. 9 is a cross section of a deployed, open-cell foam aneurysm pocket filling structure in the shape of a cylinder.

FIG. 10 is a side view of the fill structure delivery catheter having an “O”-ring connector located at the catheter's proximal end.

FIG. 11 is a helical aneurysm pocket filling structure shown at “A” in its shape when placed into the fill structure delivery tube and shown at “B” is its shape as deployed into an aneurysm pocket.

FIG. 12 illustrates an alternative embodiment of a distal portion of the fill structure delivery catheter.

### DETAILED DESCRIPTION OF THE INVENTION

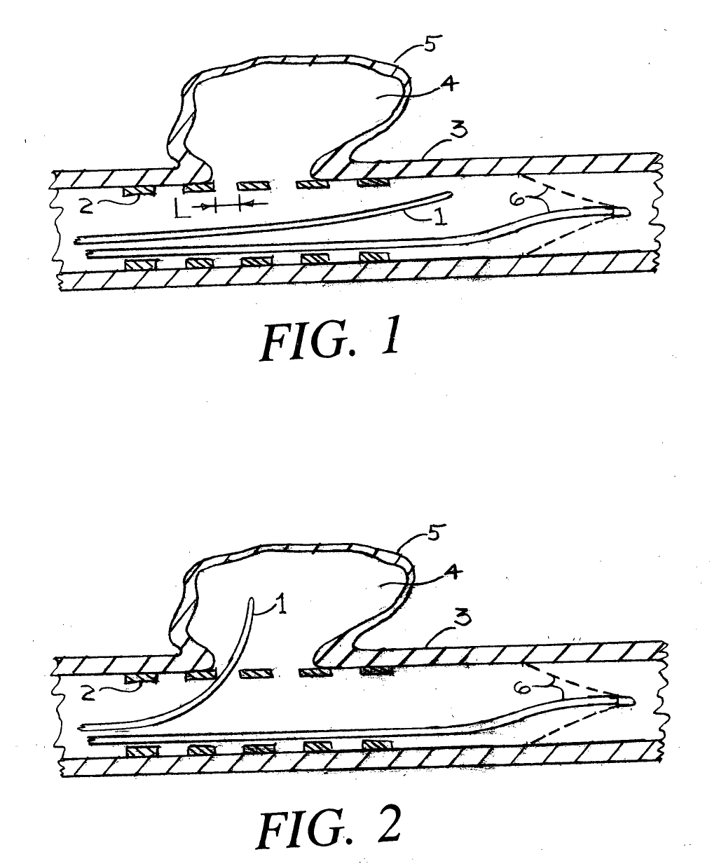

FIG. 1 is a cross section of an artery 3 onto which an aneurysm pocket 4 has formed within an aneurysm wall 5. Although the invention that is described herein is envisioned for use with any aneurysm of a human subject, the most urgent need for treatment is for aneurysms of the arterial circulation in the brain. FIG. 1 shows an aneurysm that is not ruptured. The present invention can also be used for an aneurysm that has ruptured.

FIG. 1 shows a deployed arterial filter 6 that has been placed by conventional means into the artery 3. If during the procedure to fill the aneurysm pocket with aneurysm pocket filling structures there is any inadvertent release of either tissue or aneurysm pocket filling structure(s), then the filter 6 can prevent the occurrence of a stroke which could otherwise happen. It should be understood however, that the present invention can be practiced without the placement of such an arterial filter 6.

Interventional neuroradiologists are now trained to place stents into arteries in the brain for the treatment of arterial stenoses. This is accomplished by placing an introducer sheath through the groin into the femoral artery and then advancing a stent delivery system including a stent 2 over a guide wire 1 and through the arterial system. For the present invention, the neuroradiologist (hereinafter the “operator”) would place the stent 2 with its sidewall located in a position to cover the ostium (i.e., the mouth) of the aneurysm pocket 4. After the stent 2 is deployed, the operator would then pull the guide wire 1 back and then forward so as to place the distal end of the guide wire 1 through one of the many openings in sidewall of the stent 2 as is shown in FIG. 2. The dimension “L” in FIG. 1 represents the largest diameter for a circular opening that occurs between the struts of the deployed stent 2. No aneurysm pocket filling structure that is deployed into the aneurysm pocket that has a minimum dimension that is greater than “L” can pass through the sidewall of the stent 2 and into the arterial circulation.

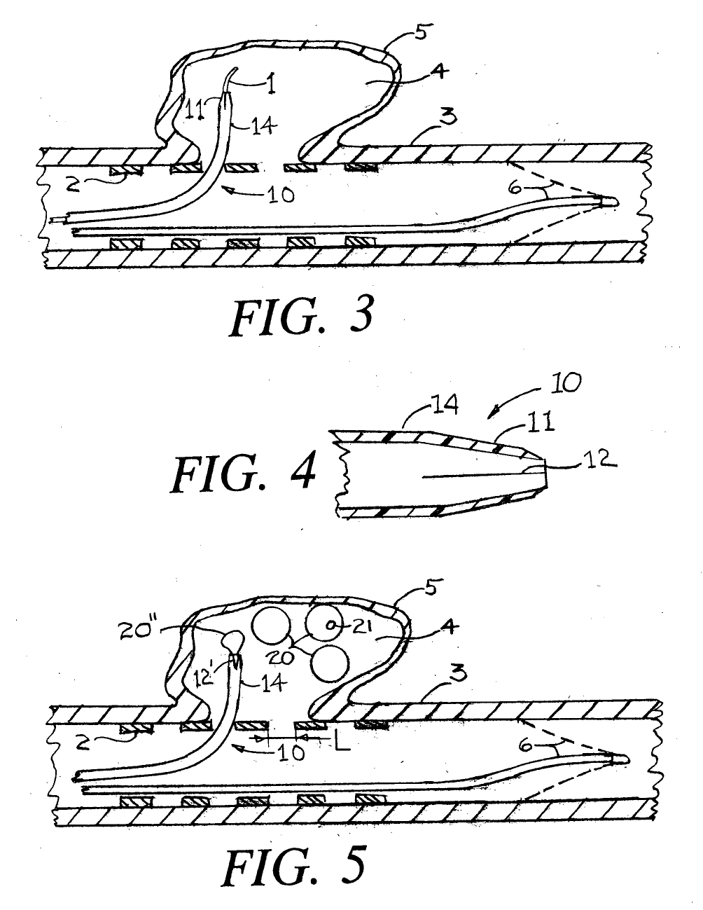

FIG. 3 shows a distal portion of a fill structure delivery catheter 10 having an elongated generally cylindrical shaft 14 being placed over the guide wire 1 so that the tapered distal end 11 having a slit 12 is situated well into the aneurysm pocket 4. It is important that a distal portion of the fill structure delivery catheter 10 is sufficiently radiopaque so that it can be clearly seen by the operator when using image-intensified fluoroscopy. It is also vitally important that the distal portion of the catheter 10 be extremely soft and flexible so that it cannot inadvertently penetrate through the thin wall 5 of the aneurysm pocket 4. Radiopacity can be accomplished by filling the plastic of the catheter 10 with a radiopaque material such as tungsten powder or barium or by the placement of a highly radiopaque metal (e.g., a ring of tantalum) onto or into the catheter 10 near or at its distal end. Softness for the distal portion can be accomplished by using a very low durometer elastomer such as polyurethane, polyethylene, silicone, etc. Ideally such a catheter would have an interior lining made from a tube of PTFE.

FIG. 4 is a longitudinal cross section of a distal portion of the shaft 14 of the fill structure delivery catheter 10. This distal portion shows the tapered section 11 that has at least one longitudinal slit 12. The purpose of the tapered section 11 is to allow for easy passage of the distal end of the catheter 10 through any opening in the sidewall of the stent 2 which covers the ostium of the aneurysm pocket 4. Although a single slot 12 may be adequate to allow passage of the compressed aneurysm pocket filling structures through the distal portion of the catheter 10, an optimum number of slots 12 would be between 2 and 4. It is also envisioned to have a uniform cylindrical lumen throughout the entire length of the fill structure delivery catheter 10 including its distal portion. Furthermore, it is envisioned that the fill structure delivery catheter 10 could have a shoulder near its distal end that has a diameter larger than “L” so that only a comparatively short distal portion of the catheter 10 can actually enter the aneurysm pocket 4. This design concept is described in detail below with the assistance of FIG. 12.

FIG. 5 shows the guide wire 1 removed and three minispheres 20 already situated within the aneurysm pocket 4. One of the minispheres 20 in FIG. 5 shows its hole 21 that is placed in the shell of each minisphere 20. Also shown in FIG. 5 is the opened slit 12′ through which a partially deployed minisphere 20″ is emerging. FIG. 5 shows a length “L” between the struts in the sidewall of the stent 2 that is placed to block the ostium of the aneurysm pocket 4. The dimension “L” is actually the diameter of the maximum diameter circle (or sphere) that could pass through the openings between the struts of the deployed stent 2. Any sphere that has a diameter that is greater than the diameter “L” would be unable to pass through the stent 2 and into the cerebral circulation. An important aspect of the present invention is that the diameter “D” of the deployed minispheres 20 should be at least 10% greater than the diameter “L”. As long as this condition is created, the sidewall of the stent 2 will prevent any minisphere 20 from embolizing into the cerebral circulation. Any such embolization would cause an ischemic stroke which, of course, would be extraordinarily unfortunate.

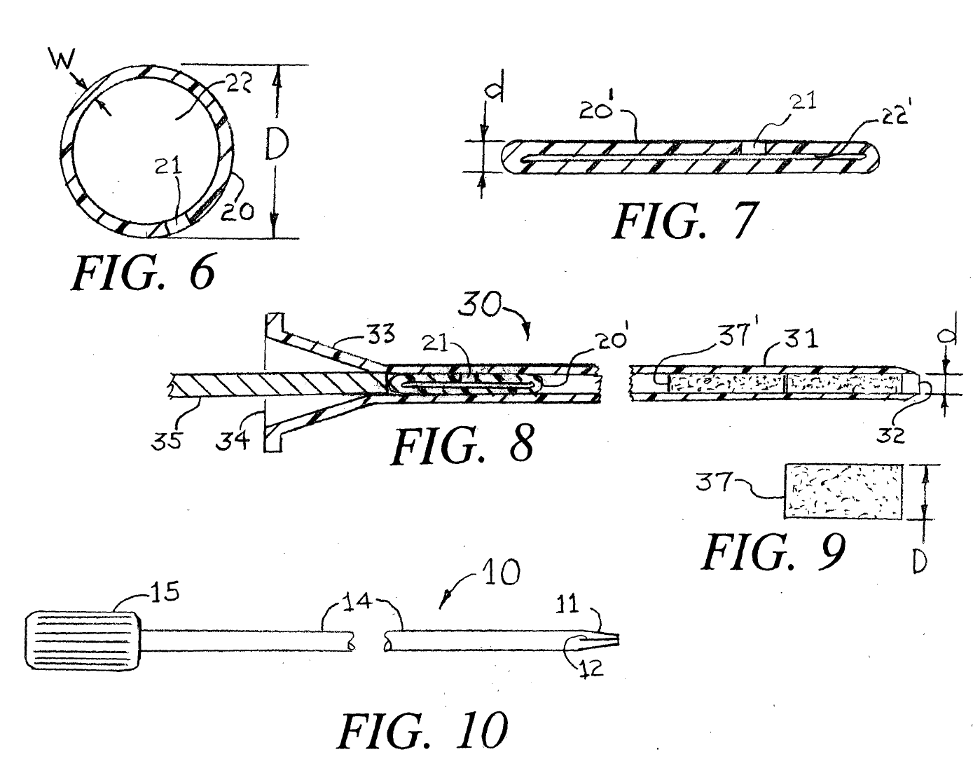

FIG. 6 shows an example of a pocket filling structure. FIG. 6 is a cross section of a typical minisphere 20 having an outside diameter “D”, a wall thickness “W”, a through hole 21 and an expanded interior volume 22. To prevent any minisphere 20 from going through the side wall of the stent 2 and into the artery 3, it is required that the diameter “D” be larger than the largest opening in the wall of the stent 2 where it covers the ostium of the aneurysm pocket 4. A minimum diameter “D” would be at least 10% greater than the largest side opening in the wall of the stent 2. Optimally, the diameter “D” would be approximately between 1.5 and 5 times the size of the maximum circular opening “L” between the struts of the deployed stent 2.

In FIG. 6 the wall thickness “W” is shown to be less than one tenth the outside diameter “D”. It is envisioned that the ratio of W/D should be between 0.01 and 0.5. The lowest ratios would be used with a solid elastomer such as a low durometer silicone rubber, latex, polyurethane, polyethylene, etc. In that case, if “D” is approximately 0.10 inches, then the wall thickness “W” would be between 0.001 and 0.010 inches. If the minisphere is formed from open-cell or closed-cell foams (such as a Nerf ball type of construction), then the wall “W” (for a “D” of 0.10) could be between 0.002 and 0.030 inches.

FIG. 7 is a highly enlarged cross section of a compressed minisphere 20′ having a through hole 21. The compressed dimension “d” must be made small enough to fit within the lumen of the fill structure storage tube 30 that is shown in FIG. 8. The diameters of the lumens of the tube 30 and the catheter 10 would also be essentially the dimension “d”. An important innovative aspect of the present invention is that the dimension “d” must of necessity be smaller than the dimension “L” when the minispheres 20′ are compressed. However, this compressed dimension “d” expands to a diameter “D” after the minispheres 20 are deployed into the aneurysm pocket 4. The minimum dimension for any shape of a deployed aneurysm pocket filling structure should be at least 10% greater than the dimension “L”. Thus a helical platinum coil having an initial compressed dimension “d” must expand radially to at least approximately 1.1 times “L” to be one embodiment of a deployed aneurysm pocket filling structure as defined for the present invention. The general rule as to the dimensions “d” and “D” compared to the dimension “L” that apply for the minisphere 20 will also apply for any other aneurysm pocket filling structure that is conceived as a design for the present invention.

Returning to FIG. 7, the compressed interior volume 22′ of the compressed minisphere 20′ will be generally in the form of a cylinder. When the minisphere 20 as seen in FIG. 6 is compressed to form the compressed minisphere 20′ as shown in FIG. 7, most of the air contained in the expanded volume 22 of FIG. 6 is pushed out through the hole 21. When the minisphere 20′ expands within the aneurysm pocket 4, it will pull blood through the hole 21 and into the expanded volume 22. This blood will then clot to form a comparatively firm structure within the aneurysm pocket 4. This is highly desirable to isolate the aneurysm pocket 4 from the artery onto which the aneurysm was formed.

FIG. 8 is a longitudinal cross section of the fill structure storage tube 30 having an elongated shaft 31, a distal opening 32 and a Luer fitting 33 with a proximal opening 34. Such storage tubes 30 could be provided to the operator with different volumes of aneurysm pocket filling structures contained therein. For example, the manufacturer could provide fill structure storage tubes 30 that contain a volume of 2.0 ml, 1.0 ml, 0.5 ml, 0.2 ml 0.1 ml, etc. The operator would select one or more of such tubes 30 to adequately fill the aneurysm pocket 4.

Also shown in FIG. 8 is one compressed minisphere 20′ situated within the lumen of the shaft 31 near the Luer fitting 33. FIG. 8 also shows a pusher rod 35 that can be used to push the compressed minispheres 20′ (or any other aneurysm pocket filling structures) through the lumens of the fill structure storage tube 30 and the fill structure delivery catheter 10. The outside diameter of the pusher rod 35 should closely match the diameter of the lumens of the fill structure storage tube 30 and the fill structure delivery catheter 10. A shoulder at a proximal portion of the pusher rod 35 (not shown) can be used to prevent the distal end of the pusher rod 35 from extending beyond the distal end of the fill structure delivery catheter 10 after the last aneurysm pocket filling structure has been placed into the aneurysm pocket 4. It is also conceived that the rod 35 would extend to near the distal end of the catheter 10 so that one or a few aneurysm pocket filling structures would still remain within the catheter 10 when the rod 35 is pushed as far forward as is possible.

FIG. 9 and the right hand portion of the fill structure storage tube 30 of FIG. 8 show cross sections of another aneurysm pocket filling structure. Specifically, FIG. 9 shows a deployed, generally cylinder aneurysm pocket filling structure 37 that could be formed in one of several different ways. For example the compressed, pre-deployed, aneurysm pocket filling structures 37′ shown within a distal portion of the tube 30 could be formed from cylindrical pellets of polyvinyl alcohol (PVA). This material in its dry state is quite hard and readily able to be pushed through the lumens of the fill structure delivery tube 30 and the fill structure delivery catheter 10, for example, using the pusher rod 35. When such pellets would enter the blood, they would quickly become a soft foam-like material that has a minimum dimension “D” as shown in FIG. 9. It is also possible to place the PVA pellet 37′ into the fill structure storage tube 8 and then add sterile water, saline solution or contrast medium so that the pellets obtain the character of a compressed foam. In this condition, they could still be pushed through the lumens of the tube 30 and the catheter 10 by means of the pusher tube 35 or by using pressurized saline solution. When they exit from the distal end of the fill structure delivery catheter 10, such foam-like structures would promptly expand with a minimum dimension “D”. Thus they could not escape from the aneurysm pocket 4. The use of contrast medium that would be absorbed within the foam has the advantage that the deployed aneurysm pocket filling structures would be radiopaque. Thus the operator could use fluoroscopy to accurately determine that the aneurysm pocket 4 was completely filled with the aneurysm pocket filling structures. Adding an anti-thrombogenic drug (e.g., heparin) to the liquid that fills the foam of the compressed aneurysm pocket filling structures 37′ would have the advantage of decreasing any propensity for the aneurysm pocket filling structures that come in contact with the sidewall of the stent to cause a blood clot. It should also be understood that the pre-deployed cylinder 37′ which forms the deployed cylindrical structure 37 could also be made from any open-cell or closed-cell elastomer foam. PVA may very well be the ideal material for an aneurysm pocket filling structure because of its well known biocompatibility and its ability to expand by as much as a factor of ten after it leaves the distal end of the fill structure delivery catheter 10.

FIG. 10 is a side view of the fill structure delivery catheter 10 which has a conical end section 11 with slit(s) 12, an elongated shaft 14 and an “O”-ring connector 15. The function of the conical section 11 has already been explained with assistance of FIG. 4. The elongated shaft 14 has a uniform interior luminal diameter which is essentially the same diameter as the luminal diameter of the fill structure storage tube 30. However, the wall of the shaft 14 may be thinner for its distal portion where it must pass through an opening in the side of the stent 2 that is placed over the ostium of the aneurysm pocket 4 and the shaft 14 can be thicker for most of the proximal length of the catheter 10. Having a thicker wall for most of the length of the fill structure delivery catheter 10 provides for additional pushability for placement of the distal end of the shaft 14 into the aneurysm pocket 4. Furthermore, as described below with the assistance of FIG. 12, an outward protruding shoulder situated near the distal end of the fill structure delivery catheter that has a diameter that is greater than the dimension “L” can prevent all but a comparatively short distal portion of the catheter 10 from entering the aneurysm pocket 4.

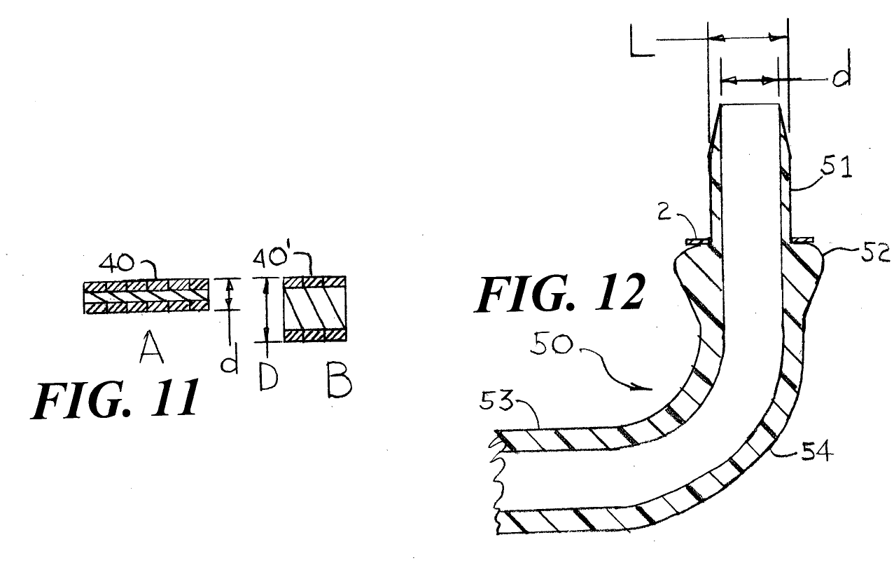

FIG. 11 illustrates an alternative embodiment of an aneurysm pocket filling structure in the form of a helix. FIG. 11 “A” shows the helix 40 in its compressed state and FIG. 11 “B” shows the deployed shape of the helix 40′. The helix 40 can be formed from either an elastomer or a radiopaque metal or a combination of two such materials. If no metal is used, then the elastomer to form the helix 40 should include a radiopaque filler (viz., barium or a metal powder) to make it radiopaque. The helix 40 is designed to be easily compressed from an initial diameter “D” to a decreased diameter “d” that can be inserted into the lumen of the fill structure storage tube 30 and through the lumen of the fill structure delivery catheter 10. The length of helix 40′ would be at least the dimension “D”. FIG. 111 is still another example of an aneurysm pocket filling structure that should have a minimum deployed dimension “D” that must be at least approximately 10% larger than the dimension “L”.

FIG. 12 is a highly enlarged, longitudinal cross section of a distal portion of an alternative embodiment of a fill structure delivery catheter 50 shown with a distal portion 51 placed through struts of the stent 2. In this FIG. 12, the relative dimensions of the struts of the stent 2 and the catheter 50 are more realistic as compared to the relative dimensions of these objects as shown in FIGS. 1-3. The fill structure delivery catheter 50 has a thick-walled section 53 and a thin-walled distal portion 51. The thicker wall section 53 is designed to provide increased pushability for the catheter 50. The thin-walled portion 51 is designed to readily fit within the space between the struts at the sidewall of the stent 2 where that stent is placed at the ostium of the aneurysm pocket 4. The decreased outer diameter of the section 51 allows easier passage of the distal end of the catheter 50 into the aneurysm pocket 2. The shoulder protrusion 52 should be highly radiopaque and is designed to allow only a very short length of the distal portion 51 from entering the aneurysm pocket 4. The radiopacity of either or both the portion 51 and the shoulder 52 provides assurance by fluoroscopy that the distal end of the catheter 15 is properly situated within the aneurysm pocket 4. The shoulder 52 could be formed from a highly radiopaque metal such as tantalum. If the inside diameter of the catheter 50 is significantly larger than the outside diameter of the guide wire 1, a hollow, flexible stylet with a tapered end (not shown) can be placed within the catheter 50 and the assembly advanced over the guide wire 1 until the distal section 51 is situated within the aneurysm pocket 4 as shown in FIG. 12. After the assembly's distal end lies within the aneurysm pocket 4, the stylet and the guide wire 2 are both withdrawn from the catheter 50. An advantage of using the catheter 50 is that there would be less resistance to pushing aneurysm pocket filling structures through the open distal end of the catheter 50 as compared to the resistance when pushing through the tapered distal end 11 of the catheter 10. Another advantage of the design shown in FIG. 12 is that the pre-formed curve 54 of the catheter 50 helps to assure that the shoulder 52 is properly aligned against the struts of the stent 2. Still another advantage of the design of FIG. 12 is that the comparatively short length of the distal portion 51 of the catheter 50 can prevent any contact between that distal portion 51 and the fragile wall 5 of the aneurysm pocket 4. An optimum length for the distal portion 51 would be less than 2 mm. It is also conceived to have a slot (not shown) in the catheter 50 located just distal to the shoulder 52. This slot could be just slightly wider than the thickness of the struts of the stent 2. Such a slot could be used to more firmly attach the distal portion of the catheter 50 to the stent 2 so that it would not fall out of the aneurysm pocket during the injection of the aneurysm pocket filling structures. The slot could be placed on only part of the circumference of the catheter 50 so that some rotation would place it onto a stent strut and additional rotation would remove the catheter 50 from that strut.

A procedure to place aneurysm pocket filling structures (e.g., cylinders formed from PVA) into an aneurysm pocket 4 would be accomplished as follows:
a) a deployed stent 2 would be placed by conventional means into a human subject so that it covers the ostium of an aneurysm pocket 4;
b) the guide wire 1 that was used to place the stent 2 or a newly placed guide wire would then have its distal end placed through the sidewall of the stent 2 and into the aneurysm pocket 4;
c) a fill structure delivery catheter 10 (or 50) would then be advanced over the guide wire 1 until its distal end was situated within the aneurysm pocket 4 and then the guide wire 1 (and possibly a hollow stylet) would be removed from the body of the human subject;
d) a fill structure storage tube 30 that was previously loaded with aneurysm pocket filling structures (e.g., compressed cylinders 37′) would have its distal end placed into the “O”-ring connector 15 of the fill structure delivery catheter 10 (or 50).
e) the “O”-ring connector 15 would then be tightened onto a distal portion of the fill structure storage tube 30 so that the lumens of the fill structure storage tube 30 and the fill structure delivery catheter 10 would be aligned;
f) either a rod 35 or a pressurized liquid (typically from a syringe) would then be used to push the aneurysm pocket filling structures through the distal end of the fill structure delivery catheter 10 and into the aneurysm pocket 4;
g) after the aneurysm pocket 4 is adequately filled with the aneurysm pocket filling structures, the fill structure delivery catheter 10 is removed from the body of the human subject.

The procedure described above could be used with the any of the aneurysm pocket fill structures that are described herein or any similar type of aneurysm pocket filling structure. Furthermore, the first step of the method could be the placement of an arterial filter just distal to the ostium of the aneurysm pocket. If that is done, then the last step would be to close the filter and remove it from the patient.

Throughout the procedure, contrast medium and fluoroscopy would be occasionally used for various purposes. Amongst the reasons for using contrast medium and fluoroscopy during this procedure would be to: 1) determine the size, shape, volume and location of the aneurysm pocket; 2) verify proper placement of the stent prior to and after stent deployment; 3) determine the position of the guide wire; 4) verify that the distal end of the fill structure delivery catheter 10 (or 50) has been accurately placed into the aneurysm pocket; 5) calculate the volume of the aneurysm pocket 4; 6) calculate the number of aneurysm pocket filling structures that are required to adequately fill the aneurysm pocket; 7) observe the release of the aneurysm pocket filling structures into the aneurysm pocket 4; 8) assist the operator in adequately filling the aneurysm pocket 4 with aneurysm pocket filling structures. Furthermore, it should be understood that a guiding catheter would typically be used to assist in advancing the stent delivery system and/or the filter and/or the fill structure delivery catheter through the patient's vascular system.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

1. A system for the percutaneous treatment of an aneurysm of an artery in a human subject, the system including:
a stent deployed with its sidewall covering the ostium of an aneurysm pocket that is formed in the wall of the artery of the human subject, the stent having a maximum circular opening diameter “L” between those stent struts that cover the ostium of the aneurysm pocket; and
a fill structure delivery system for placing aneurysm pocket filling structures into the aneurysm pocket, the fill structure delivery system including a fill structure delivery catheter that has a distal portion that is designed to be placed through the sidewall of the stent and into the aneurysm pocket, the aneurysm pocket filling structures having a compressed minimum dimension “d” when being placed through the catheter into the aneurysm pocket, the dimension “d” being expandable to a minimum dimension “D” after the aneurysm pocket filling structure is deployed into the aneurysm pocket, the dimension “D” being sufficiently larger than the diameter “L” so that the struts that form the sidewall of the stent prevent the aneurysm pocket filling structures from passing out of the aneurysm pocket and into the arterial circulation.

2. The system of claim 1 where the stent is designed to elute an anti-proliferative drug that is selected from the group containing, cytostatic drugs, sirolimus, everolimus, tacrolimus, analogs and derivatives of sirolimus, cytotoxic drugs, Taxol, paclitaxol, and analogs and derivatives of Taxol.

3. The system of claim 1 where the stent has a coating designed to decrease thrombotic activity and to reduce the incidence of subacute thrombosis.

4. The system of claim 3 where the stent is coated with phosphorocholine or heparin.

5. The system of claim 1 where the stent is designed to elute an anti-proliferative drug that is selected from the group containing, cytostatic drugs, sirolimus, everolimus, tacrolimus, analogs and derivatives of sirolimus, cytotoxic drugs, Taxol, paclitaxol, and analogs and derivatives of Taxol and the stent also has a coating that includes a drug that decreases the incidence of subacute thrombosis.

6. The system of claim 1 where the stent has a generally decreased cell size at its midsection compared to the size of the stent's cells near the ends of the stent.

7. The system of claim 1 where the stent is made from a metal that is selected from the group consisting of stainless steel, Nitinol, L605 or equivalent cobalt-chromium alloy or tantalum.

8. The system of claim 1 where the deployed aneurysm pocket filling structures have the dimension “D” that lies approximately between the dimensions 0.030 inches and 0.30 inches.

9. The system of claim 1 where aneurysm pocket filling structure is a minisphere which is a hollow spherical shell having a wall thickness “W” that lies between 0.001 and 0.020 inches.

10. The system of claim 9 where the minispheres have a hole through the spherical shell.

11. The system of claim 1 where the aneurysm pocket filling structures are formed from an elastomer.

12. The system of claim 11 where the elastomer has a comparatively low durometer.

13. The system of claim 11 where the aneurysm pocket filling structures are formed from an elastomer into which a radiopaque material has been added.

14. The system of claim 13 where the radiopaque material is selected from the group consisting of barium, contrast medium, powdered metal and powdered tungsten.

15. The system of claim 1 where each aneurysm pocket filling structures is formed from an open cell elastomer.

16. The system of claim 1 where the aneurysm pocket filling structures are coated with a material that improves their lubricity.

17. The system of claim 1 where the material of the aneurysm pocket filling structures is selected from the group consisting of a metal, part metal and part plastic, polyethylene, polyvinyl alcohol, polyurethane or silicone.

18. The system of claim 1 where the aneurysm pocket filling structures include a substance selected from the group consisting of sterile water, saline solution, contrast medium, heparin, anti-proliferative drugs or anti-thrombogenic drugs.

19. The system of claim 1 where the aneurysm pocket filling structures are formed from polyvinyl alcohol.

20. The system of claim 1 further including a fill structure storage tube as part of the fill structure delivery system, the fill structure delivery tube being designed to have compressed aneurysm pocket filling structures contained within its lumen prior to delivery of the aneurysm pocket filling structures into the aneurysm pocket.

21. The system of claim 1 where the fill structure delivery catheter has an outwardly extending shoulder located just proximal to the distal end of the fill structure delivery catheter, the shoulder having an outside diameter that is larger than the diameter “L” of opening between the struts of the stent.

22. The system of claim 1 further including a pusher rod being an elongated cylinder for most of its length, the cylinder having a diameter small enough to slide within the lumen of the fill structure delivery catheter, the pusher rod being designed to be able to push the aneurysm pocket filling structures into the aneurysm pocket.

23. The system of claim 1 further including an expandable filter that is designed to be placed into the artery of the human subject at a position that is distal to the ostium of the aneurysm pocket.

24. A method for the percutaneous treatment of an aneurysm in a human subject, the method including the following steps:
a) deploying a stent into an artery at a location where the sidewall of the stent covers the ostium of an aneurysm pocket;
b) placing a guide wire so that its distal end lies within the aneurysm pocket;
c) advancing a fill structure delivery catheter over the guide wire until its distal end lies within the aneurysm pocket;
d) placing aneurysm pocket filling structures in a compressed form into the lumen of the fill structure delivery catheter;
e) pushing the aneurysm pocket filling structures through the lumen of the fill structure delivery catheter and into the aneurysm pocket, the minimum dimension “D” of each deployed aneurysm pocket filling structure being greater than the dimension “L” which is the diameter of the largest circular opening between the struts of the sidewall of the stent that covers the ostium of the aneurysm pocket.

25. The method of claim 24 further including the step of placing a fill structure storage tube containing aneurysm pocket filling structures into a proximal portion of the fill structure delivery catheter prior to delivering the aneurysm pocket filling structures into the aneurysm pocket.

26. The method of claim 25 further including the step of placing a liquid into the lumen of the fill structure storage tube prior to placing the aneurysm pocket filling structures into the aneurysm pocket, the liquid being selected from the group consisting of contrast medium and normal saline solution.
